(19) United States
(12) Patent Application Publication
(10) Pub. No.: US 2013/0338466 A1
(43) Pub. Date: Dec. 19, 2013

(54) NERVE SURVEILLANCE ELECTRODE ASSEMBLY

(71) Applicant: Donetta Vaughan, (US)

(72) Inventors: Corbett W. Stone, San Diego, CA (US); Kabir Gambhir, San Diego, CA (US); Robin H. Vaughan, Escondido, CA (US); Brian Bowman, San Diego, CA (US); Ryan Watson, Denver, CO (US); Laura Wilson, West Chester, PA (US); Benjamin Arnold, San Diego, CA (US)

(21) Appl. No.: 13/527,334

(22) Filed: Jun. 19, 2012

(51) Int. Cl. A61B 5/04

(52) U.S. Cl. CPC A61B 5/04001
USPC 600/373

(57) ABSTRACT

A nerve surveillance electrode assembly for a surgical access instrument including a substrate, at least one electrode, at least one lead, and a connector such that the nerve surveillance electrode assembly may be selectively applied to a surgical access instrument in such a way that the nerve surveillance electrode assembly is positioned to detect nerves while the surgical access instrument is being advanced toward a surgical target site.

### BACKGROUND OF THE PRESENTLY DISCLOSED AND/OR CLAIMED INVENTIVE CONCEPTS

1. Field of the Presently Disclosed and/or Claimed Inventive Concepts

The inventive concepts disclosed and claimed herein relate to systems and methods for performing surgical procedures and, more particularly, but not by way of limitation, to systems and methods for locating nerves while creating a surgical access corridor.

2. Brief Description of Related Art

The present state of the art, when referencing a lateral surgical access approach, may consist of using the following surgical instruments: neuromonitoring probes, dilators, and retractors. After an incision is created, dilators are used to create a surgical access site which is often followed by the use of a retractor or other specialized tools to create a surgical access corridor.

During a lateral approach to a patient's spine, a psoas muscle, which is located on either side of the spine, is separated to access the spine and, in particular, an intervertebral disc space or a vertebral body within a patient's spinal column. It is desirable to avoid neural elements or nerves of the lumbar plexus that lie within the psoas muscle during such procedures. The anterior third of the psoas muscle is typically considered a safe zone for muscle separation.

The neural elements or nerves of the psoas muscle may be mapped using a stimulating probe. In this manner, the nerve free area of the psoas muscle may be located and identified. The stimulating probe may then be inserted through the psoas muscle via the nerve free tissue area or through nearly any other region, free of neural elements or nerves, toward the spine in order to initiate safe tissue separation of the psoas muscle. Dilators are next placed over the probe to create and enlarge a surgical access site. Following the use of dilators, a retractor or other specialized tools are used to further enlarge the surgical access corridor.

Surgical access instruments are now being manufactured with electrodes embedded within the body of the instrument. For example, electrodes may be provided within a dilator by manufacturing the dilator from plastic or other material capable of injection molding and providing electrically conductive elements within the walls of the dilator. Further, surgical instruments may be constructed from aluminum, or other similar metallic substance, provided with an insulation layer covering the instrument, leaving exposed regions through which energy is transferred sufficient for nerve surveillance.

While the ability to continuously monitor nerves and neural elements can be beneficial, there may be times when it is not necessary. On the other hand, many surgical instruments are still being produced without nerve surveillance electrodes.

### BRIEF DESCRIPTION OF THE DRAWINGS

FIG. 1 is an elevational view of an electrode assembly constructed in accordance with the inventive concepts disclosed herein.

FIG. 2 is a cross sectional view taken along line 2-2 of FIG. 1.

FIG. 3 is an elevational view of the electrode assembly of FIG. 1 shown attached to a dilator.

FIG. 4 is a cross sectional view taken along line 4-4 of FIG. 3.

FIG. 5 is a perspective view of a set of retractor blades shown with the electrode assembly of FIG. 1 attached to each of the retractor blades.

FIG. 6 is a cross sectional view of the electrode assembly attached to the retractor blades of FIG. 5.

FIG. 7 is an elevational view of another embodiment of an electrode assembly shown attached to a surgical access instrument.

FIG. 8 is an elevational view of another embodiment of an electrode assembly shown attached to a surgical access instrument.

FIG. 9 is an exploded, side elevational view of a portion of the electrode assembly of FIG. 8 and the surgical access instrument.

FIG. 10 is a side elevational view of the electrode assembly of FIG. 8 and the surgical access instrument.

FIG. 11 is an elevational view of another embodiment of an electrode assembly shown attached to a surgical access instrument.

FIG. 12 is an exploded view of the electrode assembly of FIG. 11 and a surgical access instrument.

FIG. 13 is a perspective view of another embodiment of an electrode assembly and a surgical access instrument.

FIG. 14 is an exploded view of the electrode assembly of FIG. 13 shown attached to a surgical access instrument.

FIG. 15 is an elevational view of another embodiment of an electrode assembly shown attached to a surgical access instrument.

FIG. 16 is an elevational view of another embodiment of an electrode assembly shown attached to a surgical access instrument.

FIG. 17 is an elevational view of the electrode assembly of FIG. 16 shown attached to a surgical access instrument.

FIG. 18 is an exploded view of another embodiment of an electrode assembly and a surgical access instrument.

FIG. 19 is a partial sectional view of the electrode assembly of FIG. 18 shown with a surgical access instrument inserted.

FIG. 20 is a partial sectional view of the electrode assembly of FIG. 18 shown attached to a surgical access instrument.

FIG. 21 is a perspective view of another embodiment of an electrode assembly shown attached to a surgical access instrument.

FIG. 22 is a perspective view of the electrode assembly of FIG. 21 shown attached to a surgical access instrument.

FIG. 23 is an elevational view of another embodiment of an electrode assembly shown attached to a surgical access instrument.

FIG. 24 is a perspective view of the electrode assembly of FIG. 23 shown attached to a surgical access instrument.

FIG. 25 is a perspective view of another embodiment of an electrode assembly shown attached to a surgical access instrument.

FIG. 26 is a perspective view of the electrode assembly of FIG. 25 shown attached to a surgical access instrument.

FIG. 27 is a partial cut away view of another embodiment of an electrode assembly.

FIG. 28 is an elevational view of another embodiment of an electrode assembly shown attached to a surgical access instrument.

FIG. 29 is an exploded view of the electrode assembly of FIG. 29 and a surgical access instrument.

FIG. 30 is a partial cut away view of the electrode assembly of FIG. 29.

FIG. 31 is an exploded view of another embodiment of an electrode assembly and a surgical access instrument.

FIG. 32 is a cross sectional view of the electrode assembly of FIG. 31.

FIG. 33 is an exploded view of another embodiment of an electrode assembly and a surgical access instrument.

FIG. 34 is a cross sectional view of the electrode assembly of FIG. 33 and attached to a surgical access instrument.

FIG. 35 is an exploded view of another embodiment of an electrode assembly and a surgical access instrument.

FIG. 36 is an elevational view of the electrode assembly of FIG. 35.

FIG. 37 is a cross sectional view taken along line 37-37 of FIG. 36.

FIG. 38 is an exploded view of another embodiment of an electrode assembly and a surgical access instrument.

FIG. 39 is an elevational view of the electrode assembly of FIG. 38.

FIG. 40 is an exploded view of another embodiment of an electrode assembly.

FIG. 41 is an elevational view of the electrode assembly of FIG. 40.

FIG. 42 is an exploded view of another embodiment of an electrode assembly and a surgical access instrument.

FIG. 43 is a partial cross sectional view of the electrode assembly of FIG. 42.

FIG. 44 is a cross sectional view of the electrode assembly of FIG. 42 and a surgical access instrument.

FIG. 45 is another cross sectional view of the electrode assembly of FIG. 42 and a surgical access instrument.

FIG. 46 is an exploded view of another embodiment of an electrode assembly and a surgical access instrument.

FIG. 47 is an elevational view of the electrode assembly of FIG. 46.

FIG. 48 is an exploded view of an electrode assembly and a surgical access instrument.

FIG. 49 is an elevational view of the electrode assembly of FIG. 48.

FIG. 50 is a cross sectional view taken along line 50-50 of FIG. 49.

### DETAILED DESCRIPTION OF EXEMPLARY EMBODIMENTS

Before explaining at least one embodiment of the presently disclosed and claimed inventive concepts in detail, it is to be understood that the presently disclosed and claimed inventive concepts are not limited in their application to the details of construction, experiments, exemplary data, and/or the arrangement of the components set forth in the following description or illustrated in the drawings. The presently disclosed and claimed inventive concepts are capable of other embodiments or of being practiced or carried out in various ways. Also, it is to be understood that the phraseology and terminology employed herein is for purpose of description and should not be regarded as limiting.

Certain exemplary embodiments of the invention will now be described with reference to the drawings. In general, such embodiments relate to nerve surveillance electrode assemblies for accessing a patient's spinal column.

As generally understood by one of ordinary skill in the art, the nerve surveillance electrode assembly will be described in connection with accessing the spine to perform a surgical procedure, but the nerve surveillance electrode assembly will find use not only in orthopaedic surgery, but in other surgical procedures in which a surgeon wishes to determine the presence, absence, or disposition of nerves or neural elements near a surgical access point or surgical target site. For example, the nerve surveillance electrode assembly may be used for anteriorly or posteriorly accessing the spine, for accessing the thoracic or cervical region of the spine, or for accessing nearly any other part of the body through which nerves or neural elements travel and are of concern. Additionally, the nerve surveillance electrode assembly may be used in the detection of neuro-muscular activation on the surface of the skin, ear-nose-throat surgery, and neuro-muscular stimulation for muscular therapy and rejuvenation.

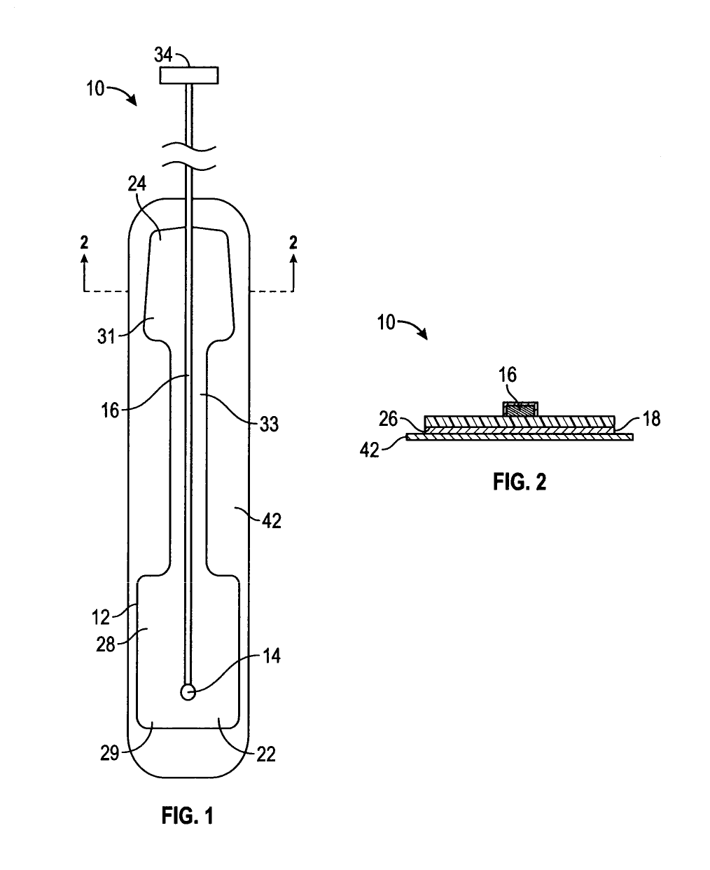

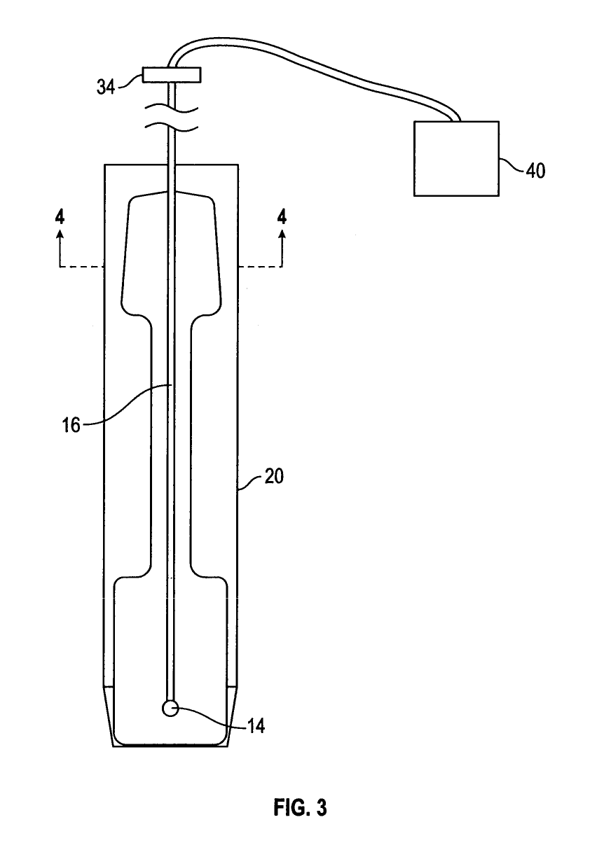

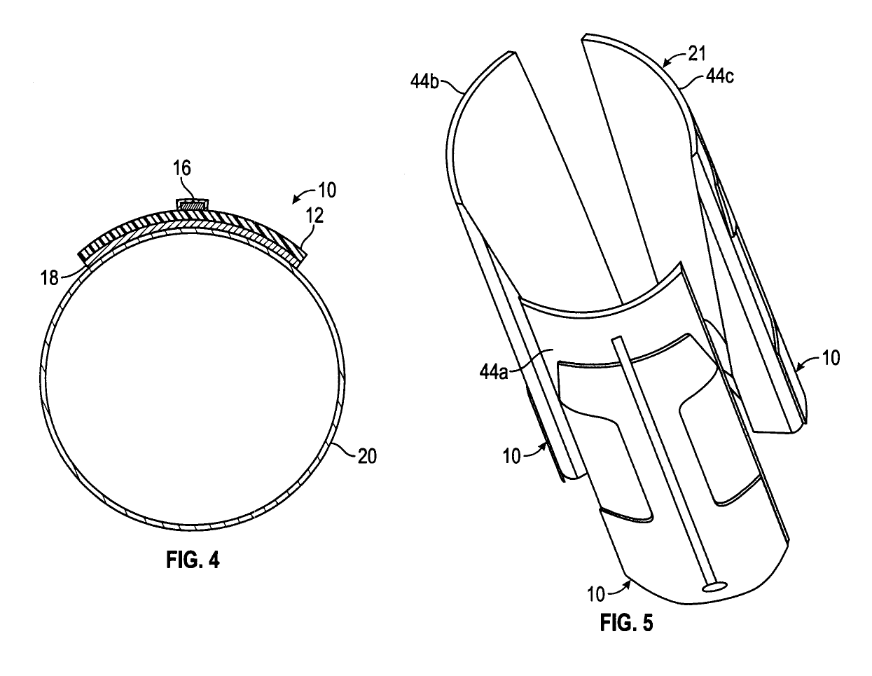

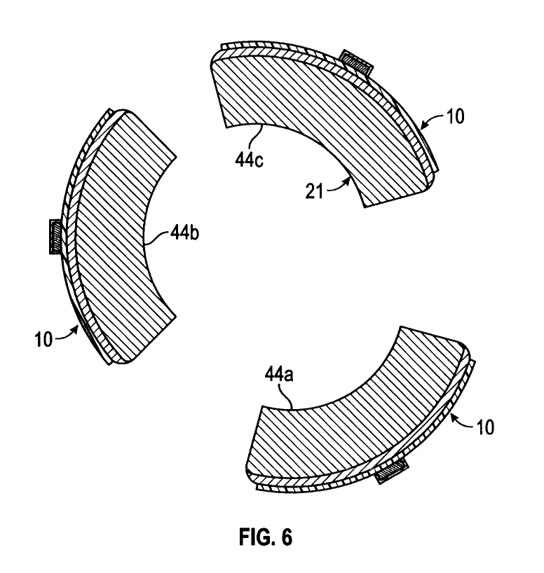

Referring now to the drawings, and more particularly to FIGS. 1-6, a nerve surveillance electrode assembly 10 is illustrated. The nerve surveillance electrode assembly 10 includes a substrate 12. The substrate 12 supports an electrode 14, a lead 16, and a connector 18 on an opposing side from the electrode 14 (FIGS. 2 and 4). The lead 16 is partially supported by the substrate 12 extending past an end of the substrate 12. The nerve surveillance electrode assembly 10 may be selectively applied to a surgical access instrument, such as a dilator 20 (FIGS. 3 and 4) and a retractor 21 (FIGS. 5 and 6), in such a way that the nerve surveillance electrode assembly 10 is positioned to detect nerves while the surgical access instrument is being advanced toward a surgical target site.

In general, the substrate 12 has a first end 22, a second end 24 opposite the first end 22, a first side 26, and a second side 28 opposite the first side 26 and is fabricated from an electrically non-conductive material. For instance, but not by way of limitation, the substrate 12 may be fabricated of polyethylene terephthalate (PET), polyvinyl chloride (PVC), polyethylene (PE), polybutylene terephthalate (PBT), or any other suitable insulating material. In one version, the substrate 12 is flexible so that the substrate 12 is conformable to the contour of the surgical access instrument when applied thereto.

The substrate 12 is illustrated as having a first enlarged portion 29, a second enlarged portion 31, and a narrow intermediate portion 33 such that the first end 22 and the first enlarged portion 29 of the substrate 12 have a width substantially corresponding to a width of the surgical access instrument. However, it should be appreciated that the substrate 12 may be formed in a variety of configurations and sizes.

The electrode 14 is positioned on the second side 28 of the substrate 12 proximate the first end 22 thereof, as shown in FIGS. 1-6. The electrode 14 can be composed of any suitable electrically conductive material, such as aluminum, platinum, platinum/iridium, stainless steel, gold, or combinations or alloys of these materials.

The lead 16 is supported by the substrate 12. In one version, the lead 16 is positioned on the second side 28 of the substrate 12. The lead 16 has a first end electrically connected to the electrode 14 and a second end which is electrically connectable to a control unit 40 (FIG. 3) in such a way as to deliver energy to the electrode 14 in an amount sufficient for nerve surveillance. To facilitate the connection of the lead 16 to the control unit 40, the second ends of the lead 16 may be provided with a coupling 34, such as a wiring harness.

The lead 16 may be in the form of an electrically conductive trace on the substrate 12. The conductive trace may be printed directly onto the substrate 12 if the substrate 12 is a dielectric. Alternatively, the conductive trace may be printed on a separate carrier sheet if the substrate is not a dielectric material. Various methods of printing electrical traces include silk screen printing, photoengraving, chemical etching, laser etching or mask. It will also be appreciated that the lead 16 may be in the form of a wire, conductive ink, conductive epoxy, or other conductor provided with an insulation coating.

In FIGS. 2, 4, and 6, the connector 18 is illustrated as being a bonding material disposed on the first side 26 of the substrate 12 such that the substrate 12 may be detachably bonded to the surgical access instrument, such as the dilator 20 and the retractor 21, with the electrode 14 positioned to detect nerves while the surgical access instrument is being advanced toward a surgical target site. The bonding material may be any suitable bonding material, such as a pressure sensitive adhesive rated for medical use.

To protect the bonding material from exposure prior to applying the nerve surveillance electrode assembly 10 to the surgical access instrument, a release sheet 42 is superimposed over the first side 26 of the substrate 12 and the bonding material. The release sheet 42 may be made of paper, coated or uncoated; plastic, flexible or rigid; or any other suitable material. The release sheet 42 is removed from the bonding material on the first side 26 of the substrate 12 of the nerve surveillance electrode assembly 10 prior to applying the nerve surveillance electrode assembly 10 to the surgical access instrument such that on removal of the release sheet, the bonding material is exposed.

FIGS. 3 and 4 illustrate the nerve surveillance electrode assembly 10 applied to the dilator 20 with the electrode 14 positioned proximate to a distal end of the dilator 20 and the lead 16 extending from a proximal end of the dilator 20. Although only one nerve surveillance electrode assembly 10 is shown detachably bonded to the surgical access instrument, such as the dilator 20, it will be understood by one skilled in the art that one or a plurality of nerve surveillance electrode assemblies 10 may be applied to the dilator 20 depending, for example, on the size and shape of the dilator 20, the density of suspected nerve and neural elements surrounding the surgical target site, and the needs of the specific surgery being performed.

The nerve surveillance electrode assembly 10 is shown applied to the retractor 21 in FIGS. 5 and 6. The retractor 21 has a plurality of retractor blades 44a-44c. One nerve surveillance electrode assembly 10 is shown applied to each of the retractor blades 44a-44c such that each nerve surveillance electrode assembly 10 may sense nerves in a different orientation than the others, allowing the sensing of nerve and neural elements in a 360° orientation around the surgical target site during the advance and expansion of the retractor 21. It will be understood by one skilled in the art that the retractor 21 may have a fewer or greater number of retractor blades and that a nerve surveillance electrode assembly 10 may be applied to any number of the plurality of the retractor blades 44a-44c.

As mentioned above, the lead 16 is connectable to a control unit 40 (FIG. 3). The control unit 40 can comprise one or more processors capable of executing processor executable code, one or more non-transitory memory capable of storing processor executable code, one or more input device, and one or more output device, all of which can be stand-alone, partially or completely network-based or cloud-based, and not necessarily located in a single physical location.

In one embodiment, the control unit 40 may include a touch screen display. In this embodiment, the touch screen display may form one of the one or more input device and one of the one or more output device. The touch screen display may be equipped with a graphical user interface (GUI) capable of communicating information to a user and receiving instructions from the user.

In use, the control unit 40 may be situated outside but close to the surgical field (such as on a cart adjacent to the operating table) such that the touch screen display is directed towards the surgeon for easy visualization. The nerve surveillance electrode assembly 10 accomplishes nerve and neural element sensing by electrically stimulating a retracted nerve root with the electrode 14 at the first end 22 of the substrate 12 when applied to the surgical access instrument while monitoring the electromyography (EMG) responses of the muscle group innervated by the particular nerve. The EMG responses provide a quantitative measure of the nerve depolarization caused by the electrical stimulus. Analysis of the EMG responses may then be used to assess the degree to which retraction of a nerve or neural element affects the nerve function over time. One advantage of such monitoring, by way of example only, is that the conduction of the nerve may be monitored during the procedure to determine whether the neurophysiology and/or function of the nerve changes (for better or worse) as the result of the particular surgical procedure. For example, it may be observed that the nerve conduction increases as the result of the operation, indicating that the previously inhibited nerve has been positively affected by the operation.

In use, the nerve surveillance electrode assembly 10 is applied to the surgical access instrument such that the electrode 14 is positioned on the substrate 12 opposite the surgical access instrument. The surgical access instrument is advanced toward the surgical target site while the electrode 14 is provided with energy sufficient to sense nerve and neural elements disposed within the tissue surrounding the surgical target site. Upon completion of a procedure for which the surgical access instrument with the applied nerve surveillance electrode assembly 10 was deployed, the surgical access instrument is removed from the surgical access site, and the nerve surveillance electrode assembly 10 may be removed from the surgical access instrument.

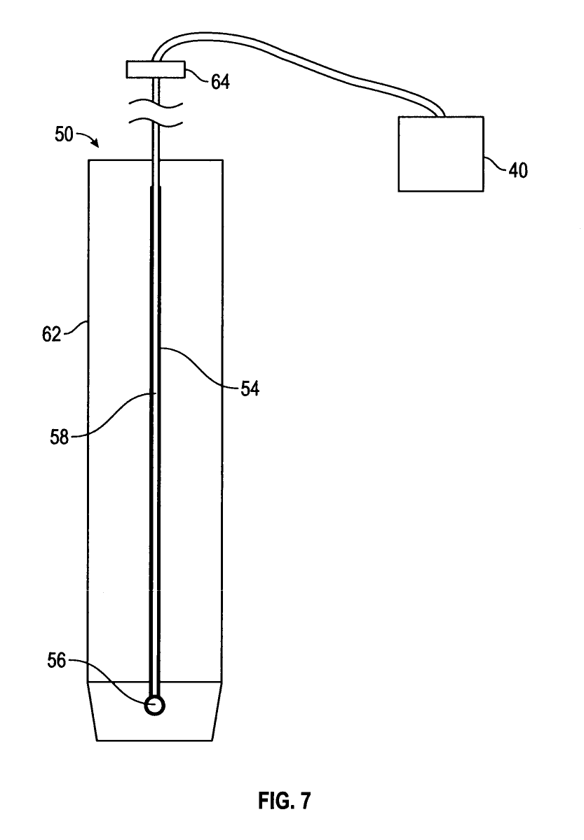

Referring now to FIG. 7, another embodiment of a nerve surveillance electrode assembly 50 is illustrated. The nerve surveillance electrode assembly 50 is substantially similar to the electrode assembly 10 described above except that the nerve surveillance electrode assembly 50 is provided with a substrate 54 sized to support an electrode 56 and a lead 58 without enlarged end portions. The substrate 54 is provided with a width substantially Furthermore, the substrate 54 is provided with a connector 60 so that the substrate 54 may be applied to a surgical access instrument, such as a dilator 62, in such a way that the nerve surveillance electrode assembly 50 is positioned to detect nerves while the surgical access instrument is being advanced toward a surgical target site.

As with the nerve surveillance electrode assembly 10, the lead 58 has a first end electrically connected to an electrode 56 and a second end which is electrically connectable to the control unit 40 in such a way as to deliver energy to the electrode 56 in an amount sufficient for nerve surveillance. To facilitate the connection of the lead 58 to the control unit 40, the second end of the lead 58 may be provided with a coupling 64, such as a wiring harness.

Also like the nerve surveillance electrode assembly 10, the connector 60 is a bonding material disposed on a first side of the substrate 54 such that the substrate 54 may be detachably bonded to the surgical access instrument, such as the dilator 62, with the electrode 56 positioned to detect nerves while the surgical access instrument is being advanced toward a surgical target site. The bonding material may be any suitable bonding material, such as a pressure sensitive adhesive rated for medical use.

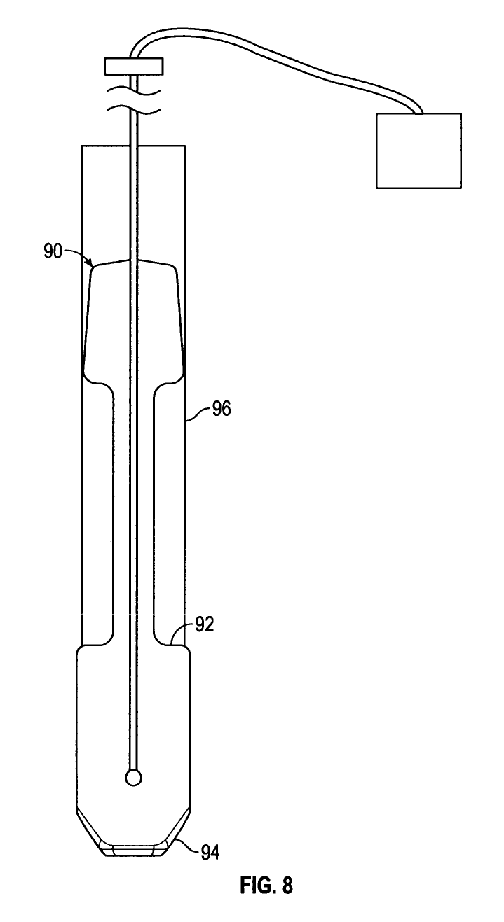

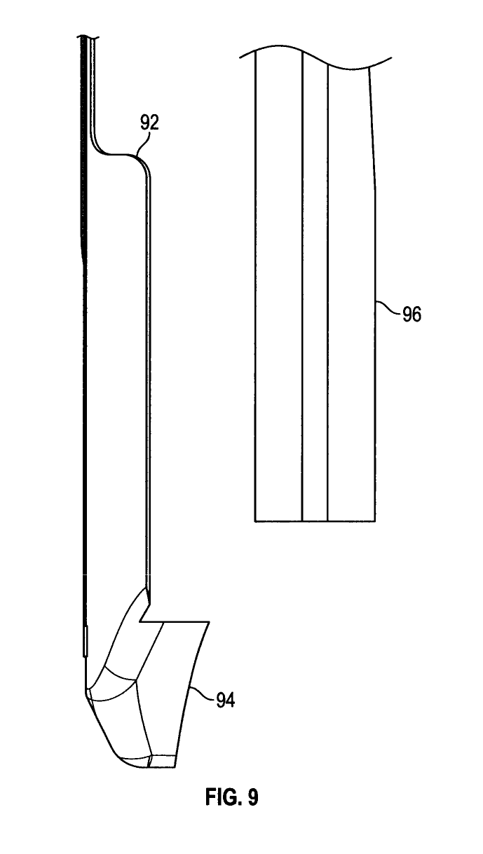

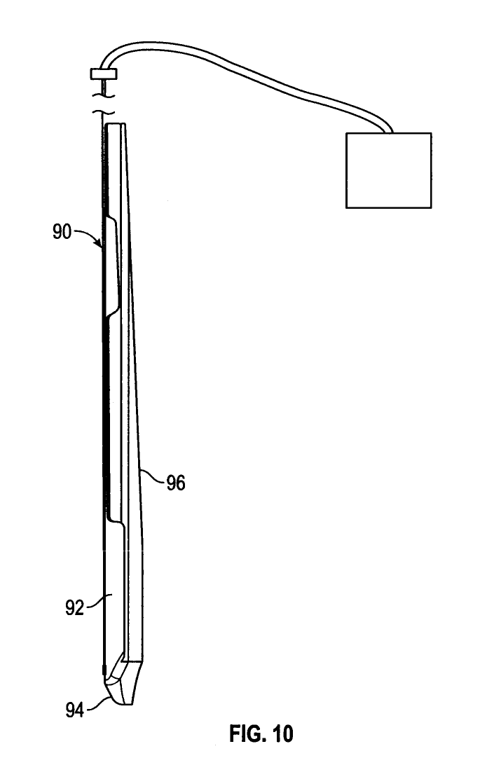

FIGS. 8-10 illustrate another embodiment of a nerve surveillance electrode assembly 90. The nerve surveillance electrode assembly 90 is substantially similar in construction and function to the nerve surveillance electrode assembly 10 described above except the nerve surveillance electrode assembly 90 has a substrate 92 provided with a nose portion 94 at a distal end thereof. The nose portion 94 is configured to mate with a distal end of a surgical access instrument, such as a retractor blade 96, and the nose portion 94 is tapered to facilitate advancement of the surgical access instrument toward a surgical target site. The distal end of the retractor blade 96 may comprise a sharp edge or a taper able to cause trauma to the surgical target site. The nose portion 94 of the substrate 92, mated to the retractor blade 96, may facilitate atraumatic entry of the retractor blade 96 to the surgical target site.

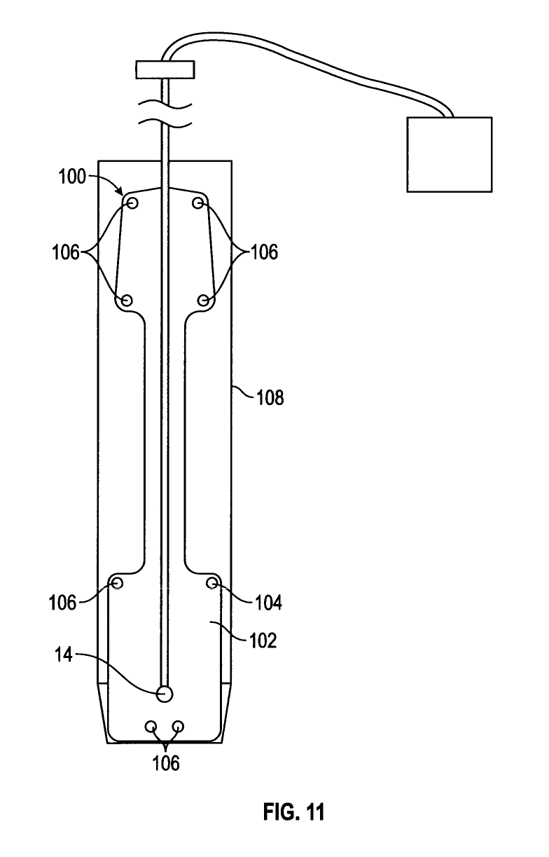

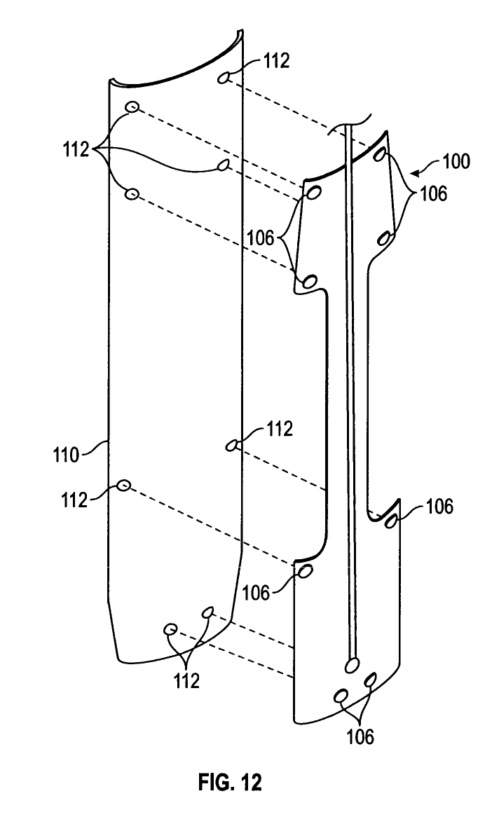

Referring now to FIGS. 11 and 12, shown therein is yet another embodiment of a nerve surveillance electrode assembly 100. The nerve surveillance electrode assembly 100 is similar to the nerve surveillance electrode assembly 10 described above except the nerve surveillance electrode assembly 100 has a substrate 102 provided with a connector 104. The connector 104 is illustrated as being a plurality of magnets 106 disposed on the substrate 102 such that the substrate 102 may be applied to the surgical access instrument, such as a dilator 108 (FIG. 11) or a retractor blade 110 (FIG. 12) constructed from a magnetic material, with the electrode 14 positioned to detect nerves while the surgical access instrument is being advanced toward a surgical target site

As shown in FIG. 12, a surgical access instrument, such as the retractor blade 110, may be constructed with a plurality of magnets 112 embedded or otherwise secured to the surgical access instrument, so as to cooperate with the magnets 106 of the substrate 102 to connect the substrate to the surgical access instrument in a self-aligning manner whereby the substrate 102 is positioned on the surgical access instrument in a predetermined position.

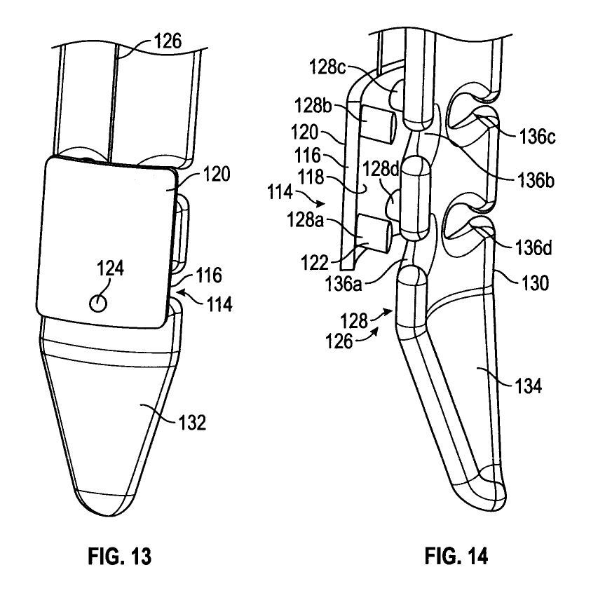

Another embodiment of a nerve surveillance electrode assembly 114 is illustrated in FIGS. 13 and 14. The nerve surveillance electrode assembly 114 includes a substrate 116 having a first side 118, a second side 120 opposite the first side 118, and a connector 122. The substrate 116 is also provided with an electrode 124 and a lead 126 supported by the substrate 116. The connector 122 is illustrated as being a plurality of protrusions 128a-128d extending from the first side 118 of the substrate 116. The protrusions 128a-128d are disposed such that the substrate 116 may engage the surgical access instrument, such as a retractor blade 130. The retractor blade 130 is constructed with a first side 132 and a second side 134 opposite the first side 132. The retractor blade 130 is constructed with recesses 136a-136d defined by the first side 132 and the second side 134. The recesses 136a-136d of the retractor blade 130 cooperate with the protrusions 128a-128d of the substrate 116 in a self-aligning manner whereby the substrate 116 engages the surgical access instrument in a predetermined position, positioning the electrode 124 is positioned to detect nerves while the surgical access instrument is being advanced toward a surgical target site.

The electrode 124 is positioned on the second side 120 of the substrate 116 proximate to a distal end thereof, as shown in FIG. 13. The electrode may be composed of similarly to electrode 14 of nerve surveillance electrode assembly 10.

The lead 126 is supported by the substrate 116, as shown in FIGS. 13 and 14, by embedding the lead 126 within the substrate 116. It will be understood by one skilled in the art, however, that the lead 126 may be supported by the second side 120 of the substrate 116. The lead 126 has a first end electrically connected to the electrode 124 and a second end which is electrically connectable to a control unit (not shown). The control unit may be implemented similarly to control unit 40 described in relation to the nerve surveillance electrode assembly 10 above. The lead 126 may be in the form of an electrically conductive trace formed on the substrate 116, a wire supported by or embedded into the substrate 116, or any other electrically conductive lead provided with an insulation coating.

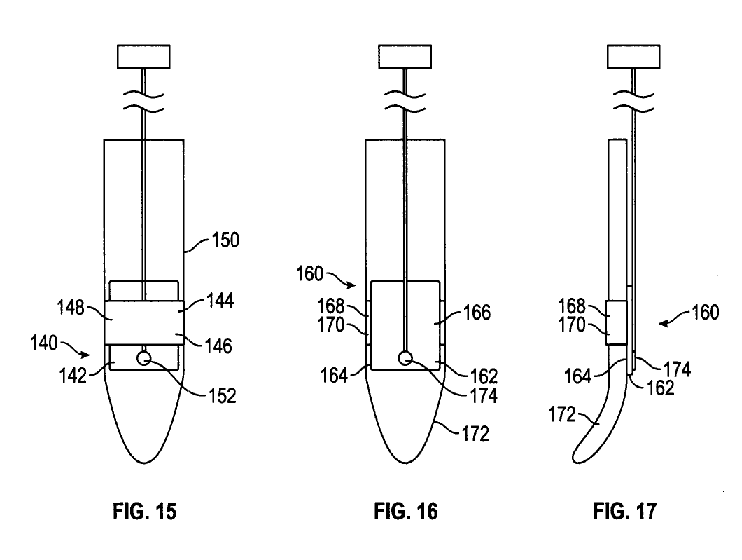

Referring now to FIG. 15, another embodiment of a nerve surveillance electrode assembly 140 is illustrated. The nerve surveillance electrode assembly 140 is substantially similar to the nerve surveillance electrode assembly 114 described above except that the electrode assembly 140 is provided with a substrate 142 having a connector 144 forming a band 146 positioned across a first side 148 of the substrate 142 and a portion of a surgical access instrument, such as a retractor blade 150. An electrode 152 is supported by the first side 148 of the substrate 142 such that when the connector 144 engages the substrate 142 and the surgical access instrument, the electrode 152 is positioned to detect nerves while the surgical access instrument is being advanced toward a surgical target site.

Referring now to FIGS. 16 and 17, shown therein is yet another embodiment of a nerve surveillance electrode assembly 160. The nerve surveillance electrode assembly 160 is substantially similar to the electrode assembly 140 described above except that the electrode assembly 160 is provided with a substrate 162 having a first side 164 and a second side 166 opposite the first side 164, and a connector 168, where the connector 168 is a band 170 secured to the first side 164 of the substrate 162 and is positioned across at least a portion of a surgical access instrument, such as a retractor blade 172. The band 170 is secured to the first side 164 of the substrate 162 by adhesive, mechanical connection, or any other suitable method of securing the band 170 to the first side 164 of the substrate 162. The band 170 positioned across at least a portion of the surgical access instrument, positions the substrate 162 such that an electrode 174 is positioned to detect nerves while the surgical access instrument is being advanced toward a surgical target site.

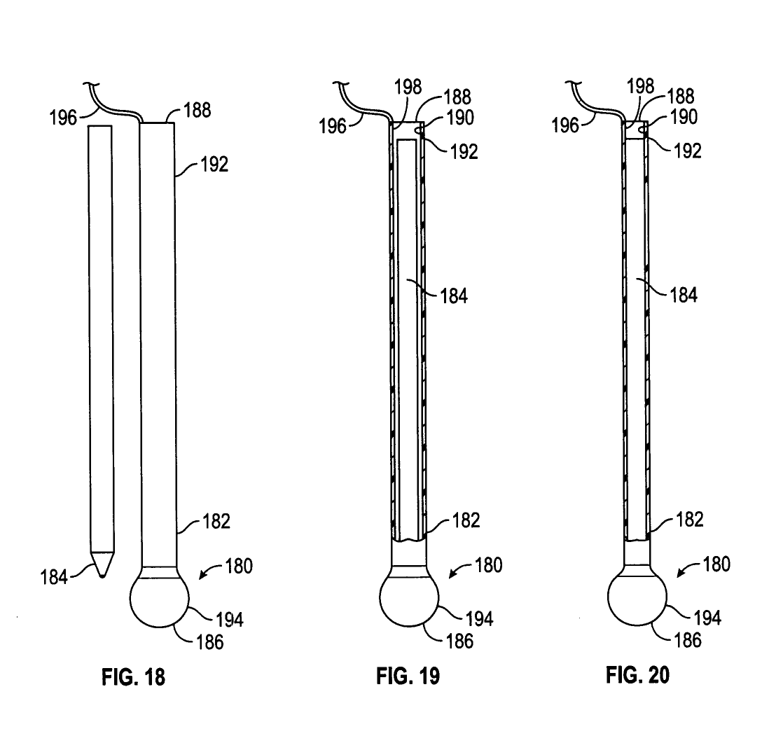

FIGS. 18-20 illustrate another embodiment of a nerve surveillance electrode assembly 180. The nerve surveillance electrode assembly 180 is provided with a substrate 182 for connecting the nerve surveillance electrode assembly 180 to a surgical access instrument, such as a probe 184. The substrate 182 is a sleeve with a first end 186 and a second end 188 opposite the first end 186, and an interior surface 190 and an exterior surface 192. An electrode 194 is disposed on the exterior surface 192 of the first end 186 of the substrate 182. Electrically connected to the electrode 194 and supported by the substrate 182 is a lead 196.

The substrate 182 is fabricated from an electrically non-conductive material capable of being contracted and remaining in the contracted stated. For instance, but not by way of limitation, the substrate 182 may be fabricated of Polyolefin, PET, or any other suitable insulating material capable of being contracted and remaining in the contracted state.

The lead 196, supported by the substrate 182, may be implemented similar to the lead 16 described in reference to nerve surveillance electrode assembly 10 above. The lead 196 may be supported along the exterior surface 192 of the substrate 182 or may be embedded within the substrate 182 and extends beyond the second end 188 of the substrate 182. The lead 196 may be electrically connected to a control unit (not shown) implemented similar to control unit 40 described above in reference to the nerve surveillance electrode assembly 10.

In use, the electrode assembly 180 is applied to the probe 184 such that the probe 184 is inserted into a void 198 defined by the interior surface 190 of the substrate 182. The substrate 182 is then contracted around the probe 184. The substrate 182 may be contracted around the probe by the application of heat, vacuum within the void 198, or any other suitable means of contracting the substrate 182 about the probe 184. Once contracted about the probe 184, the substrate 182 is positioned such that the electrode 194 is positioned to detect nerves while the probe 184 is being advanced toward a surgical target site.

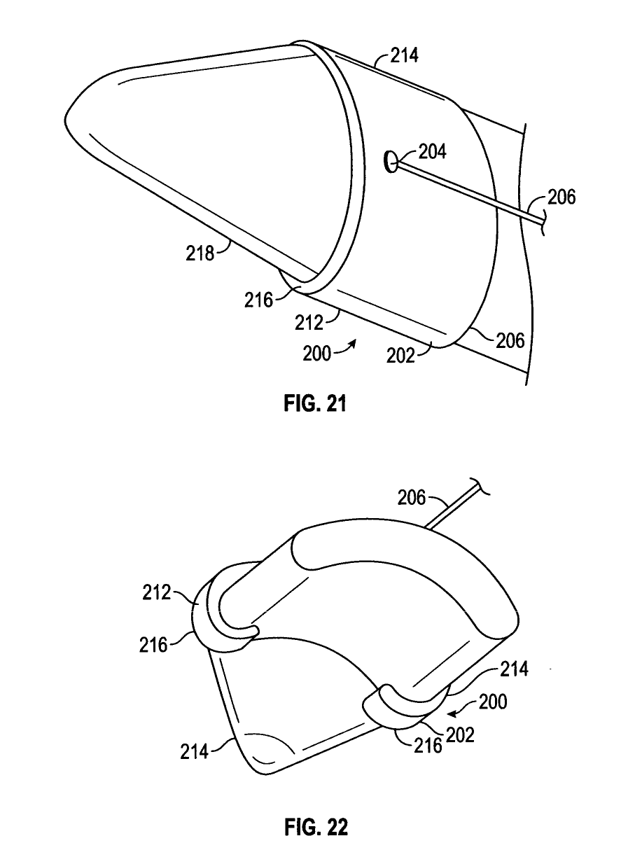

Another embodiment of a nerve surveillance electrode assembly 200 is shown in FIGS. 21 and 22. The nerve surveillance electrode assembly 200 has a substrate 202, an electrode 204 supported by the substrate 202, and a lead 206 electrically connected to the electrode 204 and supported by the substrate and extending beyond the substrate 202. The substrate 202 is provided with a first end 208, a second end 210 opposite the first end 208, a first side 212, and a second side 214 opposite the first side 212. The first side 212 and the second side 214 have a clip 216 extending from each first side 212 and second side 214. The clip 216 engages at least a portion of a surgical access instrument, such as a retractor blade 218 and when engaged positions the electrode 204 to detect nerves while the retractor blade 218 is being advanced toward a surgical target site. It will be understood by one skilled in the art that the clip 216 may extend only from one side of the substrate 202 or from both the first side 212 and the second side 214. It will also be understood by one skilled in the art that the clip 216 may be constructed of varying dimensions such that the clip 216 engages greater or lesser portions of the surgical access instrument.

The electrode 204 may be implemented similar to the electrode 14 described in reference to the nerve surveillance electrode assembly 10 above.

The lead 206 is electrically connected at one end to the electrode 204 and is supported by the substrate 202. The lead 206 may be supported on an exterior surface of the substrate 202 or embedded within the substrate 202. The lead 206 may be implemented similar to the lead 16 described above in reference to the nerve surveillance electrode assembly 10.

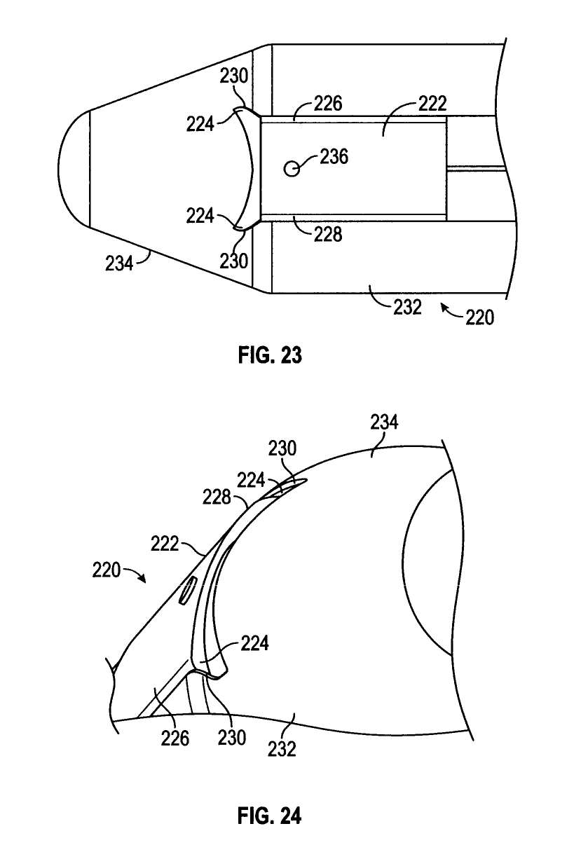

FIGS. 23 and 24 show another embodiment of a nerve surveillance electrode assembly 220. The nerve surveillance electrode assembly 220 is substantially similar to the electrode assembly 200 described above except that the electrode assembly 220 is provided with a substrate 222 having a tongue 224 extending from a first side 226 and a second side 228. The substrate 222 is sized to engage a groove 230 disposed within an exterior surface 232 of a surgical access instrument, such as a retractor blade 234. It will be understood that the substrate 222 may be constructed with one or more grooves and the surgical access instrument constructed with one or more tongues to engage the substrate 222 and the surgical access instrument. The tongue 224 engaged in the groove 230 positions the substrate 222 such that an electrode 236 is positioned to detect nerves while the surgical access instrument is being advanced toward a surgical target site. The tongue 224 engaged in the groove 230 may create a smooth continuous surface across the exterior surface 232 of the surgical access instrument.

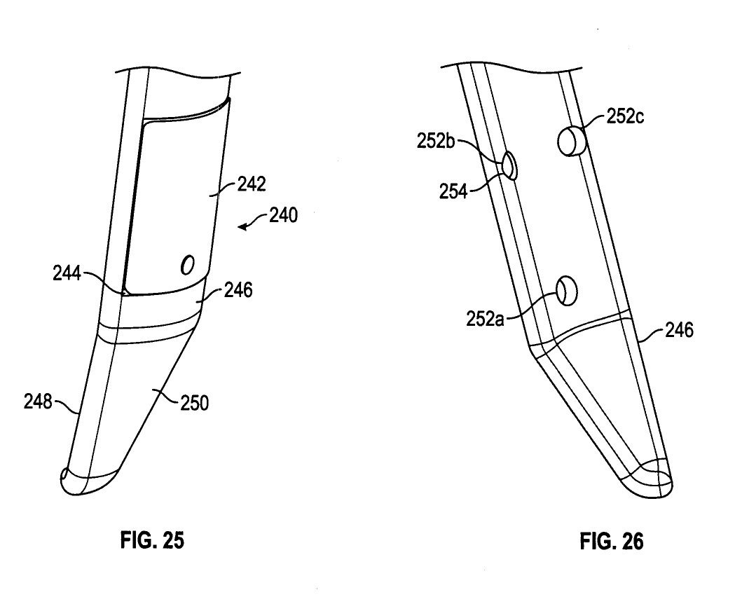

Another embodiment of a nerve surveillance electrode assembly 240 is illustrated in FIGS. 25 and 26. The nerve surveillance electrode assembly 240 is substantially similar to the electrode assembly 114 described above except that the nerve surveillance electrode assembly 240 is provided with a substrate 242 sized and shaped such that a portion of the substrate 242 may be press fit into a recess 244 of a surgical access instrument, such as a retractor blade 246. The retractor blade 246 is provided with a first side 248 and a second side 250 opposite the first side 248. The recess 244 is disposed on the second side 250 of the retractor blade 246. The recess 244 sized and shaped to cooperate with the substrate 242 of the nerve surveillance electrode assembly 240 such that a portion of the substrate 242 engages the retractor blade 246 by press fit.

The first side 248 of the retractor blade 246 is illustrated as provided with three holes 252a-252c. The three holes 252a-252c may serve as a release mechanism and pressure release for the press fit of the substrate 242 and the retractor blade 246. The three holes 252a-252c when used as a release mechanism may receive an implement suitable to pass through at least one of the three holes 252a-252c to contact a first side 254 of the substrate 242. The implement may then be used to release the friction of the press fit engagement between the substrate 242 and the retractor blade 246 by the application of force by the implement against the first side 254 of the substrate 242. The three holes 252a-252c may also serve as a pressure release during the press fit engagement of the substrate 242 and the retractor blade 246 by allowing any pressure, formed by gasses or other materials situated between the substrate 242 and the retractor blade 246 during the press fit engagement to be released through at least one of the three holes 252a-252c. It should be understood that the retractor blade 246 may be provided with any number of holes in order to facilitate release of pressure and release of the press fit engagement while remaining within the spirit and scope of the inventive concepts described herein.

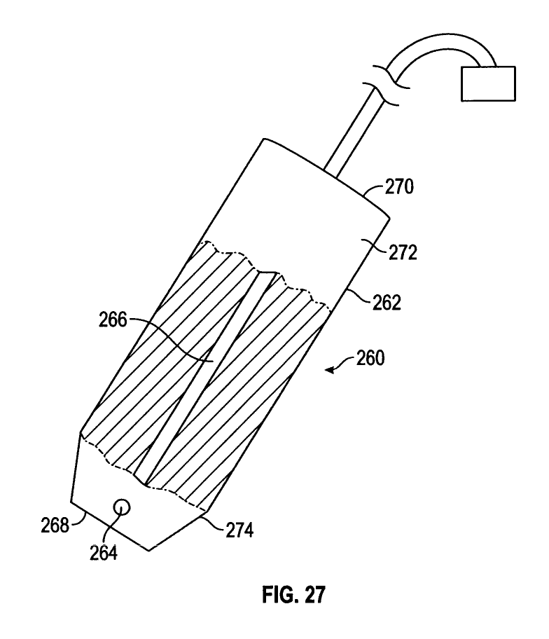

Referring now to FIG. 27, shown therein is yet another embodiment of a nerve surveillance electrode assembly 260. The nerve surveillance electrode assembly 260 is provided with a substrate 262, an electrode 264 supported by the substrate 262, and a lead 266 supported by the substrate 262. The nerve surveillance electrode assembly 260 is sized and shaped such that the substrate 262 may function as a retractor blade for a surgical access instrument in such a way that the nerve surveillance electrode assembly 260, when attached to the surgical access instrument, is positioned to detect nerves while being advanced toward a surgical target site.

The substrate 262 is provided with a first end 268 and a second end 270 opposite the first end 268, and a first side 272 and a second side 274 opposite the first side 272. The substrate 262 is fabricated from an electrically non-conductive material and may be formed during an extrusion process.

The electrode 264 is supported by the substrate 262 and may be embedded within the substrate 262. The electrode 264 is provided with a portion of the electrode 264 exposed near the first end 268 of the substrate, such that when the nerve surveillance electrode assembly 260 is attached to the surgical access instrument, the electrode 264 is positioned to detect nerves while the nerve surveillance electrode assembly 260 is being advanced toward a surgical target site.

The lead 266 is supported by and partially embedded in the substrate 262 such that a portion of the lead 266 is embedded within the substrate 262 and electrically connects to the electrode 264. A portion of the lead 266 extends from the second end 270 of the substrate 262 and is connectable to a control unit. The control unit may be implemented similarly to the control unit 40 described above in reference to the nerve surveillance electrode assembly 10.

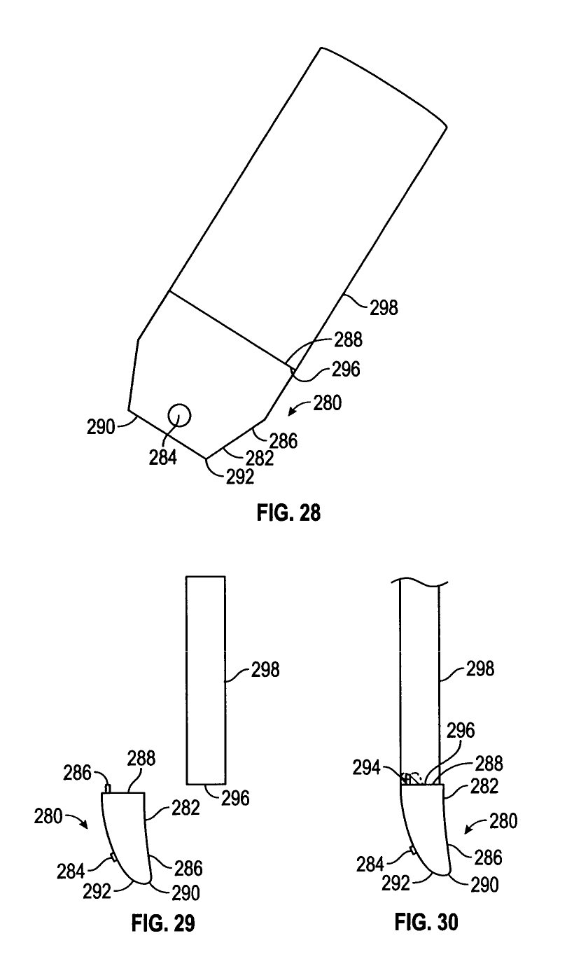

Illustrated in FIGS. 28-30 is another embodiment of a nerve surveillance electrode assembly 280. The nerve surveillance electrode assembly 280 has a substrate 282, an electrode 284 supported by the substrate 282, and a lead 286 electrically connected at one end to the electrode 284, partially supported by the substrate 282 and extending past a first end 288 of the substrate 282. The electrode 284 is partially embedded near a second end 290 of the substrate 282 such that a portion of the electrode 284 is exposed near the second end 290 of a nose portion 292 of the substrate 282 and a portion of the electrode 284 remains unexposed. The unexposed portion of the electrode 284 is electrically connected to the lead 286 which connects with a lead 294 embedded in the in a first end 296 of a surgical access instrument, for instance a retractor blade 298. When the lead 286 connects to the lead 294 the substrate 282 may engage the first end 296 of the surgical access instrument via adhesive or mechanical connection, such as magnetic, snap fit, or any other suitable mechanical connection. The lead 296 extends through the surgical access instrument and may be connectable to a control unit (not shown). The control unit may be implemented similarly to the control unit 40 described above in relation to the nerve surveillance electrode assembly 10.

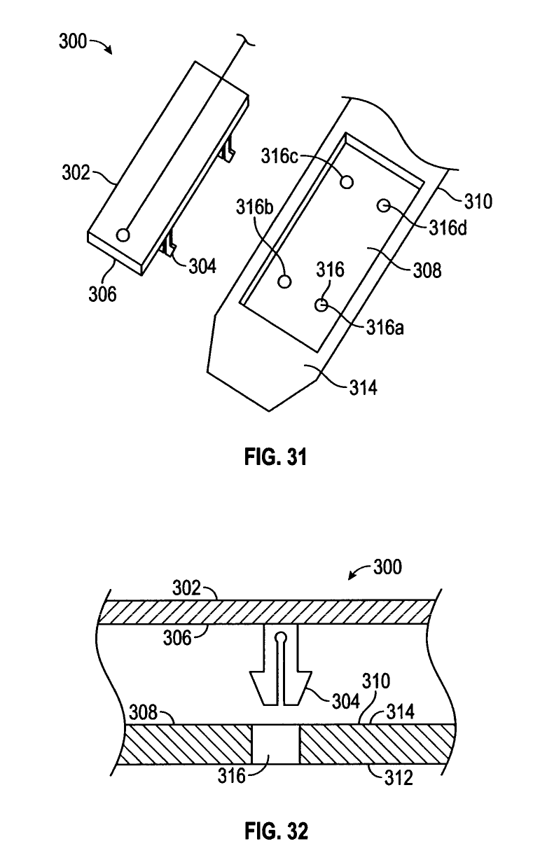

An embodiment of a nerve surveillance electrode assembly 300 is illustrated in FIGS. 31 and 32. The nerve surveillance electrode assembly 300 is substantially similar to the electrode assembly 240 described above except that the nerve surveillance electrode assembly 300 is provided with a substrate 302 which has at least one deformable snap-fit connector 304 extending from a first side 306 of the substrate 302. The substrate 302 engages a recess 308 of a surgical access instrument, such as a retractor blade 310. The retractor blade 310 is provided with a first side 312 and a second side 314 opposite the first side 312, the recess 308 extending from the second side 314 toward the first side 312 of the surgical access instrument. The recess 308 is sized and shaped to cooperate with the substrate 302 of the nerve surveillance electrode assembly 300 such that at least a portion of the substrate 302 engages the retractor blade 310 by press fit.

The second side 314 of the retractor blade 310 is illustrated as provided with four holes 316a-316d formed within the recess 308 passing through the surgical access instrument to the first side 312, creating through holes. The four holes 316a-316d serve to engage the at least one deformable snap-fit connector 304 when the substrate 302 is press fit into the recess 308 of the retractor blade 310. The at least one deformable snap-fit connector 304, while the substrate 302 is being press fit into the recess 308, deforms to pass through at least one of the holes 316a-316d. Once a portion of the at least one deformable snap-fit connector 304 passes through at least one of the holes 316a-316d, the at least one deformable snap-fit connector 304 expands to its initial shape, locking the substrate 302 in the press fit engagement with the retractor blade 310.

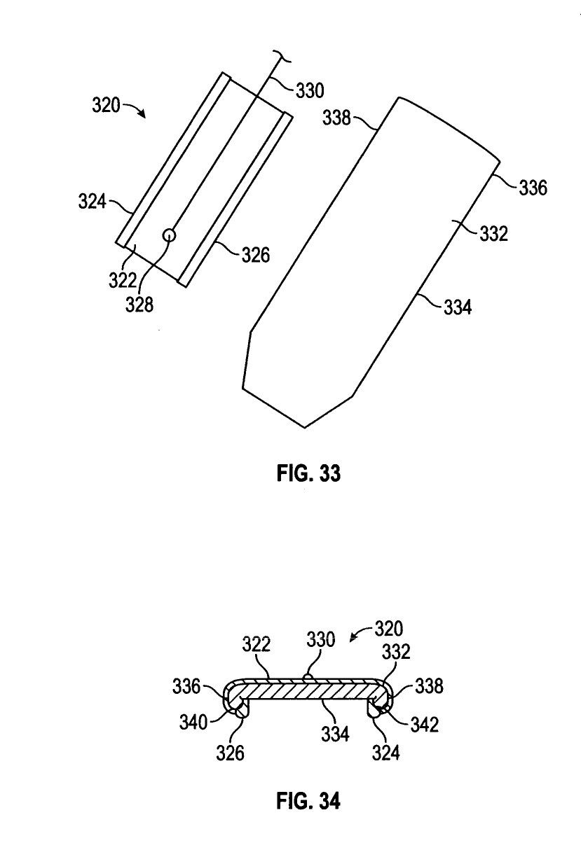

FIGS. 33 and 34 show another embodiment of a nerve surveillance electrode assembly 320. The nerve surveillance electrode assembly 320 is substantially similar to the electrode assembly 200 described above except the nerve surveillance electrode assembly 320 has a flexible film substrate 322 extending between clip members 324 and 326. The substrate 322 is provided with an electrode 328 and a lead 330 electrically connected to the electrode 328, with the electrode 328 and the lead 330 supported by the substrate 322. The substrate 322 is formed from a flexible film which is stretched across the first side 332 of a surgical access instrument, for example a retractor blade 334, such that the clip members 324 and 326 extend around a first edge 336 and a second edge 338 of the retractor blade 334 and secure the substrate 322 by engaging an inner surface 340 and 342 of the first edge 336 and the second edge 338, respectively.

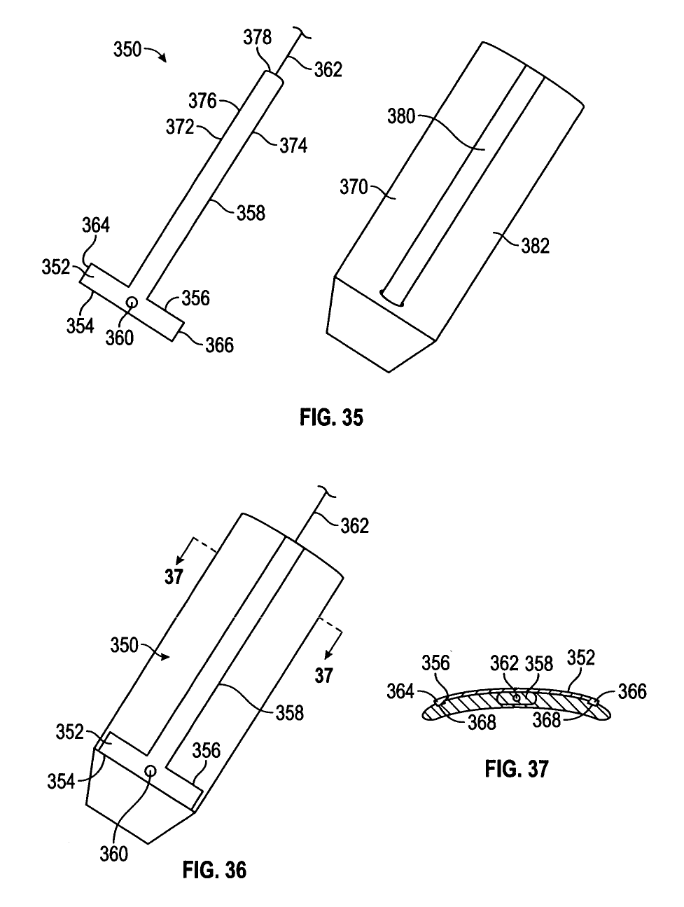

FIGS. 35-37 show yet another embodiment of a nerve surveillance electrode assembly 350. The nerve surveillance electrode assembly 350 is similar to the nerve surveillance electrode assembly 200 described above except the nerve surveillance electrode assembly 350 has a substrate 352 which has first end 354, a second end 356, opposite the first end 354, and a tail 358 extending from the second end 356. The substrate 352 is provided with an electrode 360 supported by the substrate 352 and a lead 362 supported by the substrate 352 and extending beyond the tail 358 of the substrate 352. The substrate 352 also has a first side 364 and a second side 366, opposite the first side 364. A clip 368 extends from each of the first side 364 and the second side 366. The clip 368 engages at least a portion of a surgical access instrument, such as a retractor blade 370. It will be understood that the clip 368 may engage a side of the retractor blade 370 opposite the substrate 352 when positioned on the retractor blade 370 or may engage the same side as that engaged by the substrate 352.

The tail 358 has a tongue 372 extending from each of a first side 374 and a second side 376, and a first end 378. The tail 358 is sized to engage a groove 380 disposed within an exterior surface 382 of the retractor blade 370. It will be understood that the tail 358 may be constructed with one or more grooves and the retractor blade 370 with one or more tongues to engage the tail 358. The tongue 372 engaged in the groove 380 positions the tail 358 and substrate 352 such that the electrode 360 is positioned to detect nerves while the retractor blade 370 is being advanced toward a surgical target site. The tongue 372 engaged in the groove 380 may create a smooth continuous surface across the exterior surface 382 of the retractor blade 370.

The lead 362 is embedded within and supported by the substrate 352 and tail 358 extending from an electrical connection with the electrode 360 through the substrate 352 and tail 358, and extending past the first end 378. It will be appreciated by one skilled in the art that the lead 362 may be supported by an exterior surface of the substrate 352 and tail 358.

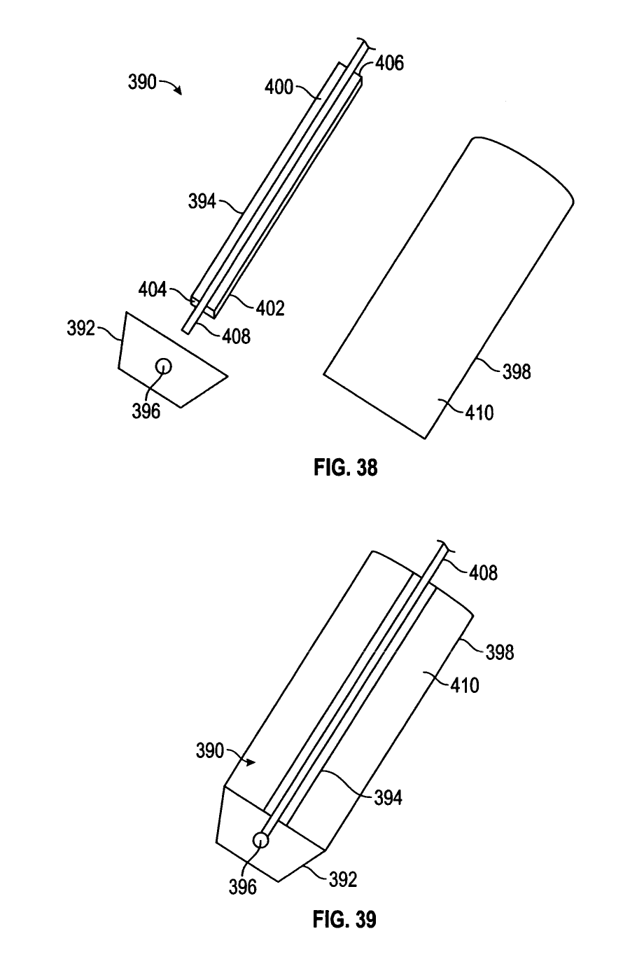

Referring now to FIGS. 38 and 39, illustrated therein is another embodiment of a nerve surveillance electrode assembly 390. The nerve surveillance electrode assembly 390 is similar to the nerve surveillance electrode assembly 280 described above except that the nerve surveillance electrode assembly 390 is provided with a first substrate 392 and a second substrate 394. The first substrate 392 is provided with an electrode 396 and is configured to mate with a distal end of a surgical access instrument, such as a retractor blade 398, and tapered to facilitate advancement of the surgical access instrument toward a surgical target site. The first substrate 392 may engage the distal end of the retractor blade 398 by a mechanical connection, such as a magnetic connection or a press fit.

The second substrate 394 has a first side 400 and a second side 402 opposite the first side 400, and a first end 404 and a second end 406 opposite the first end 404. The second substrate 392 supports a lead 408 along the first side 400 of the second substrate 394. The second substrate 394 is provided with an adhesive on the second side 402 to connect the second substrate 394 to the retractor blade 398. The lead 408 extends beyond each of the first end 404 and second end 406.

In use, the first substrate 392 and second substrate 394 are connected to the retractor blade 398 on an exterior surface 410 of the retractor blade 398. The lead 408 extending beyond the first end 404 is electrically connected to the electrode 396 of the first substrate 392.

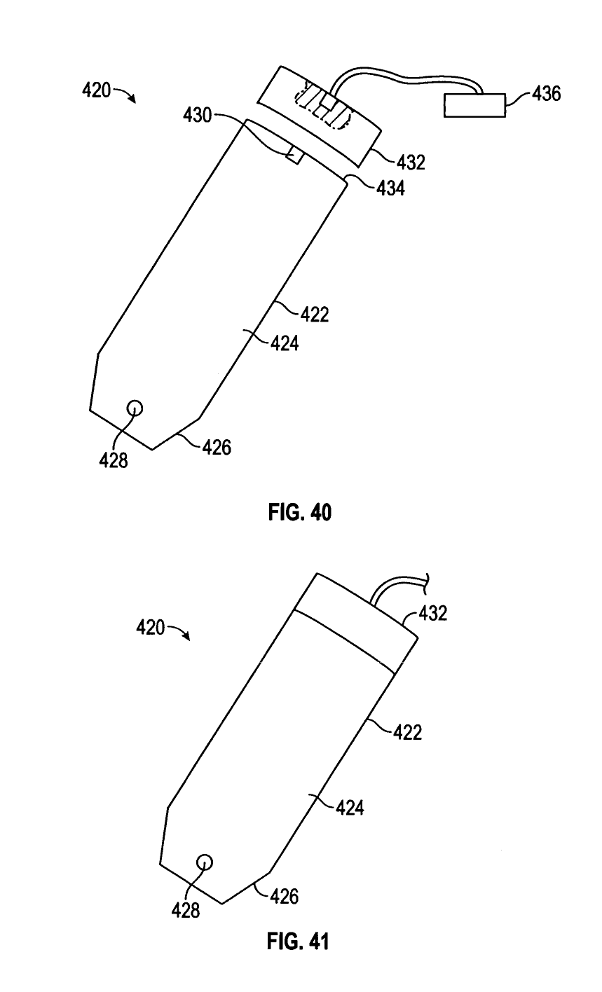

FIGS. 40 and 41 show another embodiment of a nerve surveillance electrode assembly 420. The nerve surveillance electrode assembly 420 is substantially similar to the electrode assembly 260 described above except that the nerve surveillance electrode assembly 420 is provided with a substrate 422 formed from an anodized coating 424. The anodized coating 424 is applied to a surgical access instrument, such as a retractor blade 426. The retractor blade 426 supports an electrode 428 and a lead 430 which is electrically connected to the electrode 428. The anodized coating 424 is applied to the retractor blade 426 and over a portion of the lead 430, leaving exposed the electrode 428 and a portion of the lead 430. An end cap 432 configured to engage a proximal end 434 of the retractor blade 426 electrically connects the exposed portion of the lead 430 to a control unit 436. The control unit 436 may be implemented similarly to control unit 40 described above in reference to the nerve surveillance electrode assembly 10.

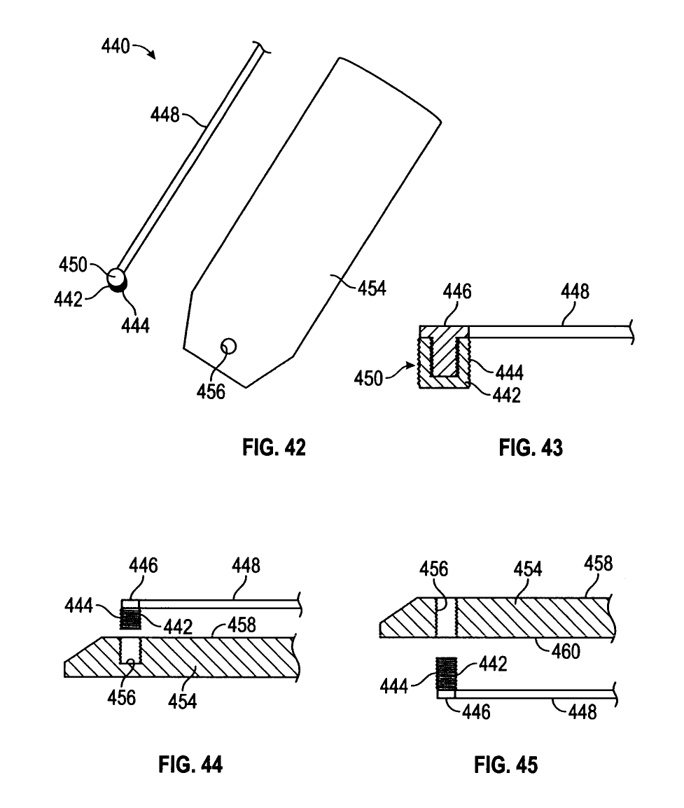

An embodiment of a nerve surveillance electrode assembly 440 is illustrated in FIGS. 42-45. The nerve surveillance electrode assembly 440 has provided with a substrate 442 comprised of a set screw 444, an electrode 446 housed within the set screw 444, and a lead 448 electrically connected to the electrode 446 and extending outwardly therefrom. The set screw 444 is formed from an outer insulating body 450 and an inner conductive body 452 which forms the electrode 446.

The lead 448, electrically connected at a first end to the electrode 446, provides energy sufficient for the electrode 446 to detect nerves while being directed toward a surgical access site. The lead 448 may be connectable at a second end to a control unit (not shown) to provide energy to the electrode 446 through the lead 448 sufficient to detect nerves while being directed toward a surgical access site. The control unit (not shown) may be implemented similarly to the control unit 40 described above in reference to the nerve surveillance electrode assembly 10.

In use, the set screw 444 is inserted into a surgical access instrument, such as a retractor blade 454. The retractor blade 454 may be provided with a hole 456 to receive the set screw 444. The hole 456 may be provided in a first surface 458 of the retractor blade 454. The hole 456 may also be provided as a through hole extending between the first surface 458 and a second surface 460 of the retractor blade 454. The set screw 444 may be inserted into the hole 456 through either the first surface 458 or the second surface 460 of the retractor blade 454 such that the electrode 446 is positioned to detect nerves while directed toward a surgical target site. The set screw 444 received in the hole 456 provided on the first surface 458 positions the electrode 446 such that the electrode 446 is exposed on the first surface 458 of the retractor blade 454, as shown in FIG. 44. The set screw 444 received in the hole 456 through the second surface 460, as shown in FIG. 45, positions the electrode 446 on the first surface 458 of the retractor blade 454, as the electrode 446 is exposed in FIG. 45 on the opposing side from the lead 448.

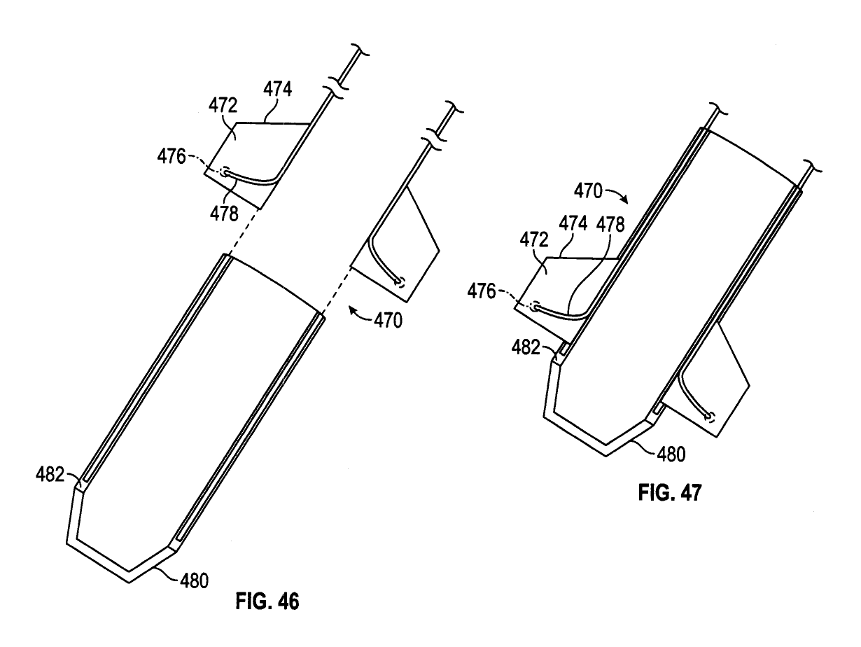

FIGS. 46 and 47 show yet another embodiment of a nerve surveillance electrode assembly 470. The nerve surveillance electrode assembly 470 has a substrate 472 provided with a winglet portion 474, an electrode 476 supported by the substrate 472, and a lead 478 electrically connected to the electrode 476 and partially supported by the substrate 472. The winglet portion 474 is configured to engage a surgical access instrument, such as a retractor blade 480. The winglet portion 474 protrudes from a first side 482 of the retractor blade 480. As shown in FIGS. 46 and 47, the nerve surveillance electrode assembly 470 may engage either side of the retractor blade 480, and in use one or more nerve surveillance electrode assemblies 470 may be used simultaneously on a single retractor blade 480. The winglet portion 474 of the substrate 472, engaging the retractor blade 480 may facilitate detection of nerves and neural elements further from the retractor blade than possible when an electrode is mounted closer to the retractor blade 480. Further, the winglet portion 474 may hold tissue away from the surgical access site. The winglet portion 474 may engage the retractor blade 480 via tongue and groove, magnetic, press-fit, adhesive, hinge, rotating pivot, or any other suitable connection.

The electrode 476, supported by the substrate 472 may be implemented similarly to the electrode 14 described above in reference to the nerve surveillance electrode assembly 10.

The lead 478, electrically connected to the electrode 476 at a first end, may have a supported portion, supported by the substrate 472, and an unsupported portion. In use the unsupported portion of the lead 478 may travel along a groove provided within the first side 482 of the retractor blade 480, if the nerve surveillance electrode assembly 470 engages the retractor blade 480 via a tongue and groove connection mechanism. The lead 478 extends from the electrical connection with the electrode 476 and may be connectable at a second end to a control unit (not shown). The control unit (not shown) may be implemented similarly to the control unit 40 described above in reference to the nerve surveillance electrode assembly 10.

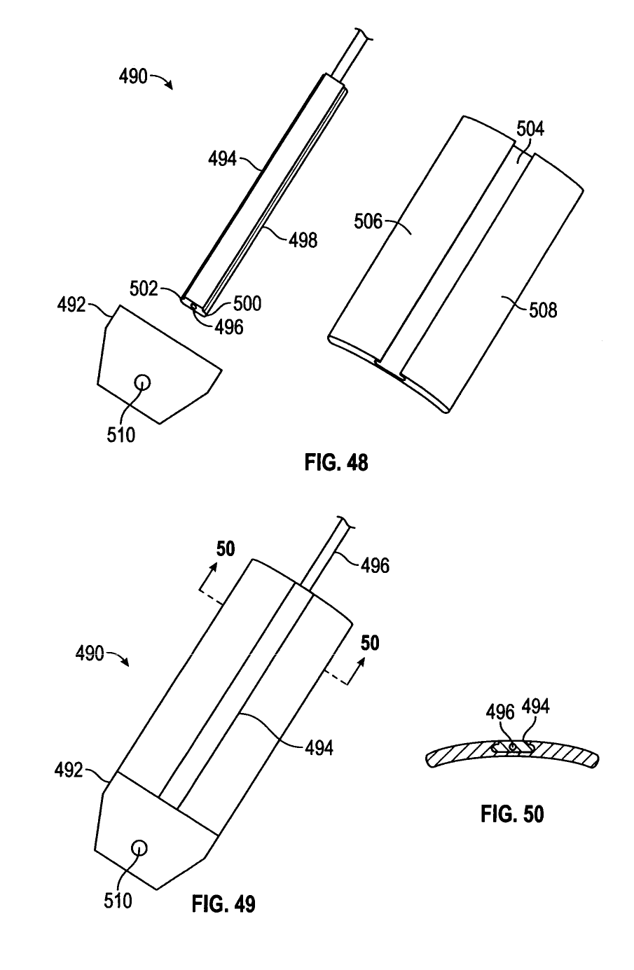

Another embodiment of a nerve surveillance electrode assembly 490 is illustrated in FIGS. 48-50. The nerve surveillance electrode assembly 490 is substantially similar to the electrode assembly 390 described above except that the nerve surveillance electrode assembly 490 is provided with a first substrate 492 and a second substrate 494, the second substrate 494 provided with a lead 496 embedded within the body of the second substrate 494 and configured to engage a surgical access instrument via tongue and groove. The second substrate 494 has tongue 498 extending from a first side 500 and a second side 502. The second substrate 494 is sized to engage a groove 504 disposed within an exterior surface 506 of a surgical access instrument, such as a retractor blade 508. It will be understood that the second substrate 494 may be constructed with one or more grooves and the surgical access instrument constructed with one or more tongues to engage the second substrate 494. The tongue 498 engaged in the groove 504 positions the second substrate 494 such that the lead 496 embedded within the second substrate 494 may form an electrical connection with an electrode 510 provided in the first substrate 492. The tongue 498 engaged in the groove 504 may create a smooth continuous surface across the exterior surface 506 of the retractor blade 508.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A nerve surveillance electrode assembly for a surgical access instrument, comprising:
a substrate having a first end, a second end opposite the first end, a first side, and a second side opposite the first side, the substrate fabricated of an electrically insulating material;
an electrode positioned on the second side of the substrate proximate the first end of the substrate;
a lead supported by the substrate with a first end electrically connected to the electrode and a second end being connectable to a control unit in such a way as to deliver energy to the electrode in an amount sufficient for nerve surveillance; and
means for connecting the substrate to the surgical access instrument with the electrode positioned to detect nerves while the surgical access instrument is being advanced toward a surgical target site.

2. The electrode assembly of claim 1, wherein the means for connecting is a bonding material disposed on the first side of the substrate.

3. The electrode assembly of claim 2, wherein the bonding material is a pressure sensitive adhesive.

4. The electrode assembly of claim 2, further comprising a release sheet superimposed over the first side of the substrate so that upon removal of the release sheet the bonding material is exposed.

5. The electrode assembly of claim 1, wherein the means for connecting comprises at least one magnet supported by the substrate.

6. The electrode assembly of claim 1, wherein the means for connecting comprises at least one protrusion extending from the first side of the substrate so as to be engageable with the surgical access instrument.

7. The electrode assembly of claim 1, wherein the means for connecting is a band positionable across the second side of the substrate and at least a portion of the surgical access instrument.

8. The electrode assembly of claim 1, wherein the means for connecting is a band positionable across at least a portion of the surgical access instrument, the band secured to the first side of the substrate.

9. The electrode assembly of claim 1, wherein the means for connecting is the substrate.

10. The electrode assembly of claim 9, wherein the substrate is a sleeve and wherein the sleeve is contractible.

11. The electrode assembly of claim 9, wherein the substrate has at least one clip extending from the substrate engageable with at least a portion of the surgical access instrument.

12. The electrode assembly of claim 1, wherein the means for connecting is a clamp positionable across at least a portion of the substrate and positionable across at least a portion of the surgical access instrument.

13. The electrode assembly of claim 1, wherein the means for connecting is a tongue extending from the substrate engageable with the surgical access instrument.

14. The electrode assembly of claim 1, wherein a second substrate supports a portion of the lead.

15. The electrode assembly of claim 1, wherein the lead is embedded within the substrate.

16. The electrode assembly of claim 1, further comprising a coupling attached to the second end of the lead.

17. The electrode assembly of claim 1, wherein the substrate is flexible so that the substrate is conformable to a contour of the surgical access instrument.

18. A nerve surveillance electrode assembly in combination with a surgical access instrument, comprising:
the surgical access instrument; and
a nerve surveillance electrode assembly comprising:
a substrate having a first end, a second end opposite the first end, a first side, and a second side opposite the first side, the substrate fabricated of an electrically insulating material,
at least one electrode positioned on the second side of the substrate proximate the first end of the substrate,
a lead supported by the substrate with a first end electrically connected to the electrode and a second end being connectable to a control unit in such a way as to deliver energy to the electrode in an amount sufficient for nerve surveillance, and
wherein the substrate is connected to the surgical access instrument with the electrode positioned to detect nerves while the surgical access instrument is being advanced toward a surgical target site.

19. The electrode assembly in combination with the surgical access instrument of claim 18, wherein the connection comprises a recess disposed within a second side of the surgical access instrument with at least one hole disposed within the surgical access instrument communicating between a first side of the surgical access instrument and the recess, wherein at least a portion of the substrate is engageable with the recess disposed within the second side of the surgical access instrument.

20. The electrode assembly in combination with the surgical access instrument of claim 18, wherein the connection comprises at least one protrusion extending from the first side of the substrate engageable with at least one recess disposed on at least one side of the surgical access instrument.

21. The electrode assembly in combination with the surgical access instrument of claim 18, wherein the connection is a tongue extending from the substrate engageable with a groove disposed in a surface of the surgical access instrument.

22. A method of accessing a surgical target site while protecting adjacent nerves, comprising:
obtaining an electrode assembly comprising:
a substrate having a first end, a second end opposite the first end, a first side, and a second side opposite the first side, the substrate fabricated of an electrically insulating material;
at least one electrode positioned on the second side of the substrate proximate the first end of the substrate; and
a lead supported by the substrate with a first end electrically connected to the electrode and a second end being connectable to a control unit in such a way as to deliver energy to the electrode in an amount sufficient for nerve surveillance;
attaching the first side of the substrate to a surgical access instrument such that the electrode is positioned to detect nerves while the surgical access instrument is being advanced toward a surgical target site;
advancing the surgical access instrument toward the surgical target site; and
sensing the presence of a nerve with the electrode.

23. The method of claim 22 further comprising:
removing the surgical access instrument from the surgical target site; and
detaching the substrate of the electrode assembly from the surgical access instrument.

24. The method of claim 20 wherein the attaching step further comprises conforming the substrate to a contour of the surgical access instrument.
